(19) United States
(12) Patent Application Publication
(10) Pub. No.: US 2015/0112254 A1
(43) Pub. Date: Apr. 23, 2015

(54) MEDICAL BALLOON WITH MULTI-POSITION ACTUATOR FOR PRECISELY ARRANGING THE WORKING SURFACE

(71) Applicant: CLEARSTREAM TECHNOLOGIES LIMITED, Enniscorthy, County Wexford (IE)

(72) Inventors: Angela Kay Jensen, Tempe, AZ (US); Andrew Schaffer, Tempe, AZ (US); Corey E. Stapleton, Gilbert, AZ (US)

(21) Appl. No.: 14/383,770

(22) PCT Filed: Mar. 8, 2013
(86) PCT No.: PCT/US13/29987

§ 371 Date: Sep. 8, 2014

Related U.S. Application Data
(60) Provisional application No. 61/608,917, filed on Mar. 9, 2012.
(60) Provisional application No. 61/747,444, filed on Dec. 31, 2012.

(30) Foreign Application Priority Data
Mar. 9, 2012 (NL) 20008452

(51) Int. Cl. A61M 25/10

(52) U.S. Cl. CPC A61M 25/10; A61M 25/104; A61M 2025/1079
USPC 604/103.1

(57) ABSTRACT

A medical balloon may be provided with an actuator. In one embodiment a balloon catheter includes a shaft extending in a longitudinal direction, said shaft having a proximal end and a distal end, and supporting at least one radiopaque identifier. An inflatable balloon supported along the distal end of the shaft, the balloon when inflated including a working surface. An actuator is provided for aligning at least one end of the working surface with the at least one radiopaque identifier. In another embodiment, the actuator assists with preferential folding. Related methods are also disclosed.

The following U.S. Patent Applications are incorporated herein by reference: 61/608,852; 61/608,859; 61/608,862; 61/608,897; 61/608,902; 61/608,908; 61/608,913; 61/608,917; 61/608,927; 61/608,932; 61/608,941; and 61/747,444.

### TECHNICAL FIELD

This disclosure relates generally to balloons for performing medical procedures, such as angioplasty and, more particularly, to a medical balloon having a predetermined portion, such as a working surface or folds, that may be precisely actuated during use.

### BACKGROUND OF THE INVENTION

Balloons are routinely used to resolve or address flow restrictions or perhaps even complete blockages in tubular areas of the body, such as arteries or veins. In many clinical situations, the restrictions are caused by hard solids, such as calcified plaque, and require the use of high pressures to compact such blockages. Commercially available balloons employ complex technology to achieve high pressure requirements without sacrificing the profile of the balloon. Besides high pressure requirements, the balloons should also be resistant to puncture, easy to track and push, and present a low profile, especially when used for angioplasty.

In clinical practice, angioplasty balloons are expanded from a deflated, folded state to an expanded state within a vessel to treat a target area, such as a portion of the circumferential inner wall I of a blood vessel V, as shown in FIGS. 1 and 2. The inflation is traditionally completed using an X-ray contrast agent to provide better visibility under X-ray or other form of radiography during the interventional procedure, as illustrated in FIGS. 3 and 3a (which shows a balloon 12 with a balloon wall 28 bounding a contrast media CM having a width DX and bombarded with radiation R over a fluroscopic detector plate FDP and a graph illustrating the resulting image that may be obtained). Typically, a 70/30 percent mixture of contrast agent and saline is used to inflate the balloon during an angioplasty procedure.

The physician performing the angioplasty procedure should be able to locate the position of the uninflated balloon with accuracy, so that the balloon will be properly positioned once inflated. This is conventionally accomplished by attaching marker bands on the catheter shaft in the region corresponding to the balloon working surface. This “working surface” is the surface along the portion of the balloon that is used to achieve the desired treatment effect, such as contacting the calcified plaque (which surface in the case of a balloon having conical or tapering sections at the proximal and distal ends is typically co-extensive with a generally cylindrical barrel section).

Misalignment of the marker bands during placement along the shaft sometimes results in their failure to correspond precisely to the extent of the working surface, as is shown in FIG. 4 (note misalignment amount X between each interior marker band M carried by shaft S and working surface W of balloon 12, which also typically includes a radiopaque tip P at the distal end). Even upon exercising great care to position the markers properly on the underlying shaft in alignment with anticipated boundaries of the working surface when the balloon is inflated, there remains a tendency for mismatch due to several possible factors. One such factor may be the tolerance stack-ups arising as a consequence of the affixation of the balloon to the distal end of the catheter shaft. The balloon also has a tendency to grow in the longitudinal direction when inflated, especially with large and particularly long balloons. Another factor is the tendency of the portion of the catheter shaft within the balloon to bend or flex during inflation. This may lead to misalignment between radiopaque markers fixed to the shaft and the working surface.

Whatever the cause, the resulting misalignment may prevent the clinician from accurately identifying the location of the working surface of the balloon during an interventional procedure. This may lead to a geographic misplacement, or “miss,” of the intended contact between the target area T and the working surface W of the balloon 12 (see FIG. 2). It is especially desirable to avoid such an outcome when the balloon is designed to deliver a payload (such as a therapeutic agent (e.g., a drug, such as paclitaxel, rapamycin, heparin and the like), a stent, a stent graft, or combinations of the foregoing) or a working element to a specified location within the vasculature, since a miss may prolong the procedure (such as, for example, by requiring redeployment of the balloon 12 or the use of another balloon catheter in the case of a drug coated balloon).

Upon deflation, the balloon may also be subject to a phenomenon known as “pancaking.” In this condition, the balloon 12 folds down upon itself to a flattened state, as shown in FIG. 5. This situation may cause the balloon to be viewed through fluoroscopy as perhaps still being in the inflated condition, since the full width of the balloon may still be perceived. This can give the clinician the false perception that the balloon remains inflated, when in fact it is not.

Accordingly, the need is identified for a balloon for which the working surface may be identified during an interventional procedure with enhanced precision. The solution would take into account the possible mismatch between fixed locations on the catheter shaft and the balloon to define the working surface, and would operate independent of the position of the portion of the catheter shaft within the balloon. The balloon may also be designed to fold in a preferential manner, which may help to avoid it from assuming undesirable configurations. Overall, procedural efficiency would be enhanced without remarkably increasing cost or complexity, and in a manner that can be applied to many existing catheter technologies without extensive modification.

### SUMMARY OF THE INVENTION

An object of the disclosure is to provide a balloon for which the working surface may be identified during an interventional procedure with enhanced precision.

A further object of the disclosure is to provide a balloon that folds in a preferential manner.

### BRIEF DESCRIPTION OF THE DRAWINGS

FIGS. 1-9 are illustrative of the background of the invention;

FIGS. 10 and 11 illustrate a first embodiment according to the disclosure;

FIG. 12 illustrates another aspect of the disclosure;

FIGS. 13 and 14 illustrate a second embodiment according to the disclosure;

FIGS. 15, 16 and 17 illustrate a third embodiment according to the disclosure; and

FIGS. 18-22 illustrate a fourth embodiment of the disclosure.

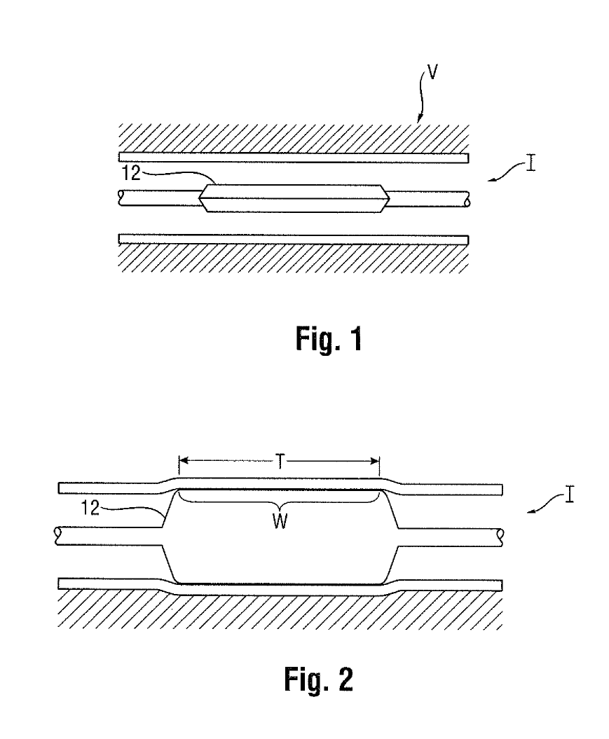

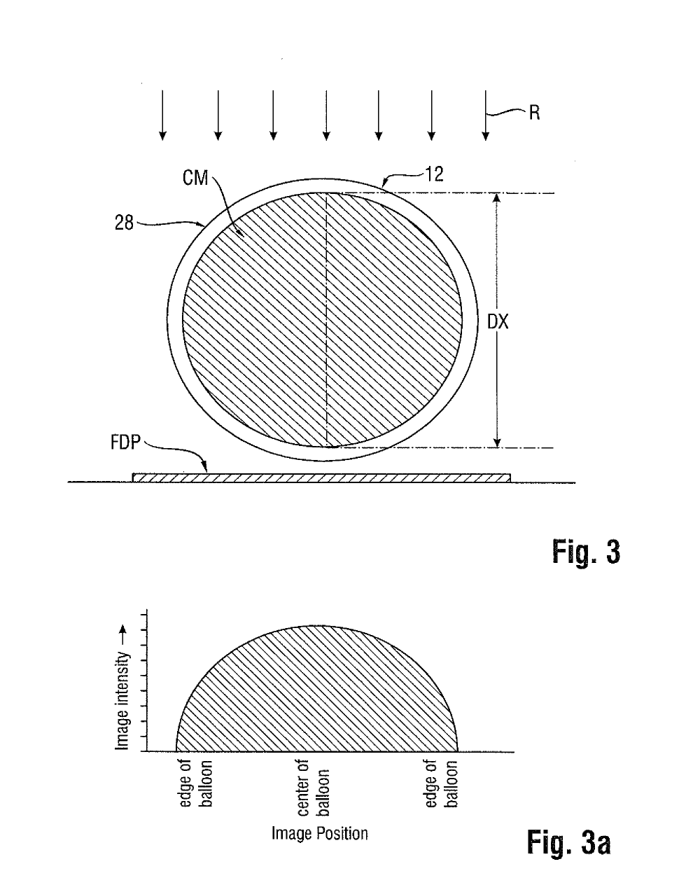

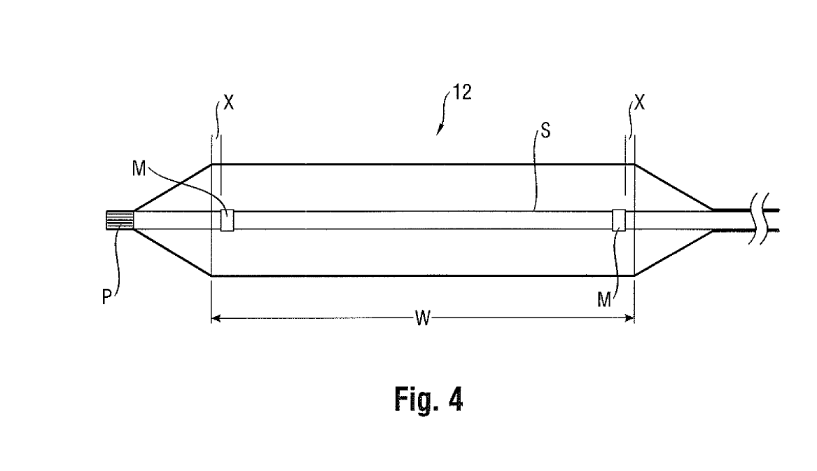

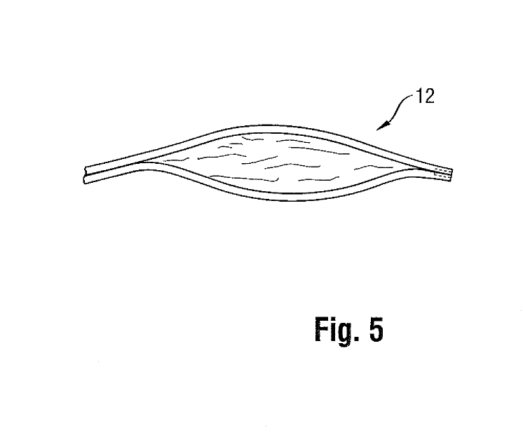

### MODES FOR CARRYING OUT THE INVENTION

The description provided below and in regard to the figures applies to all embodiments unless noted otherwise, and features common to each embodiment are similarly shown and numbered.

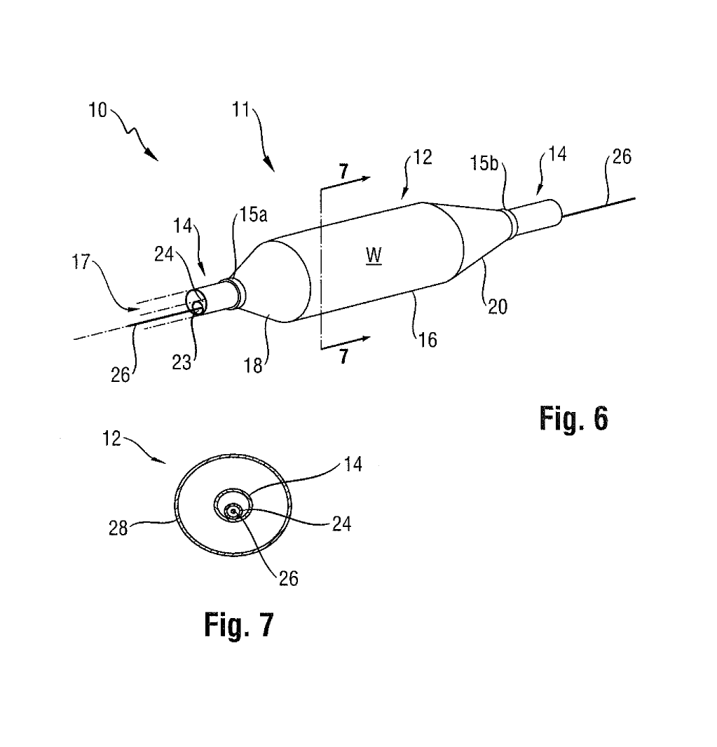

Provided is a catheter 10 having a distal portion 11 with a balloon 12 mounted on a catheter tube 14. Referring to FIGS. 6, 7, and 8, the balloon 12 has an intermediate section 16, or “barrel,” and end sections 18, 20. In one embodiment, the end sections 18, 20 reduce in diameter to join the intermediate section 16 to the catheter tube 14 (and thus sections 18, 20 are generally termed cones or cone sections). The balloon 12 is sealed at balloon ends (proximal end 15a and distal end 15b ) on the cone sections 18, 20 to allow the inflation of the balloon 12 via one or more inflation lumens 17 extending within catheter tube 14 and communicating with the interior of the balloon 12.

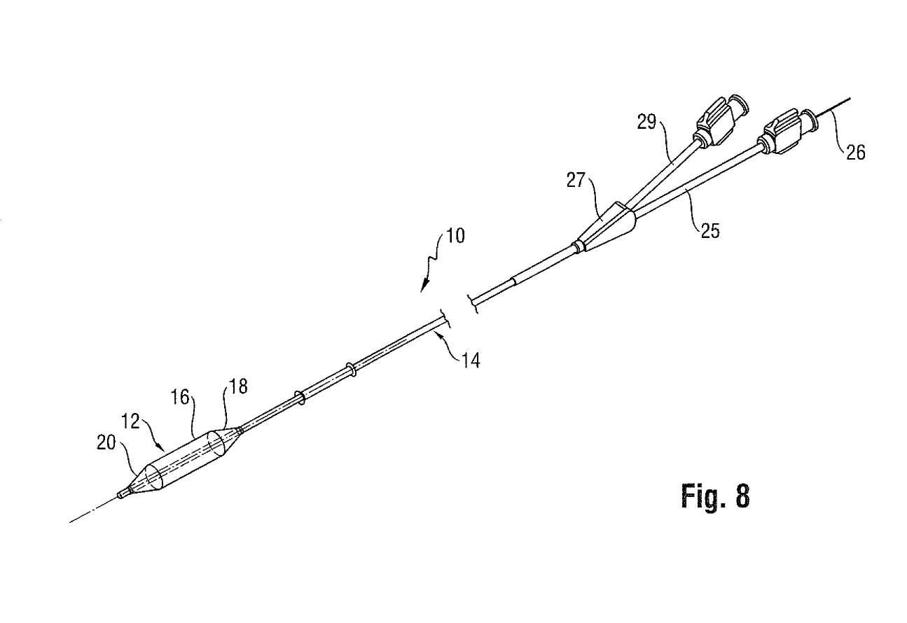

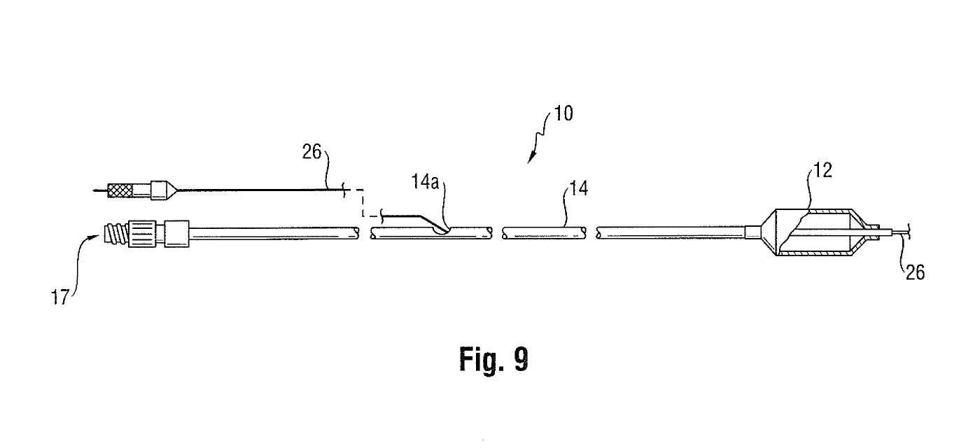

The catheter tube 14 also includes an elongated, tubular shaft 24 forming a guidewire lumen 23 that directs the guidewire 26 through the catheter 10, and along the distal end of which the balloon 12 may be located. As illustrated in FIG. 8, this guidewire 26 may extend through the proximal end of the catheter 10 and a first port 25 of a connector 27 into the lumen 23 to achieve an “over the wire” (OTW) arrangement, but could also be provided in a “rapid exchange” (RX) configuration, in which the guidewire 26 exits a lateral opening 14a closer to the distal end (see FIG. 9) or else is fed through the tip distally of the balloon 12 (not shown). A second port 29 may also be associated with catheter 10, such as by way of connector 27, for introducing a fluid (e.g., saline, a contrast agent, or both) into the interior compartment of the balloon 12 via the inflation lumen 17.

Balloon 12 may include a single or multi-layered balloon wall 28 forming the interior for receiving the inflation fluid. The balloon 12 may be a non-compliant balloon having a balloon wall 28 that maintains its size and shape in one or more directions when the balloon is inflated. Examples of non-compliant balloons may be found in U.S. Pat. No. 6,746,425 and Publication Nos. US 2006/0085022, US 2006/0085023 and US 2006/0085024, the disclosures of which are hereby incorporated herein by reference. The balloon 12 in such case also has a pre-determined surface area that remains constant during and after inflation, also has a pre-determined length and pre-determined diameter that each, or together, remain constant during and after inflation. However, the balloon 12 could be semi-compliant or compliant instead, depending on the particular use.

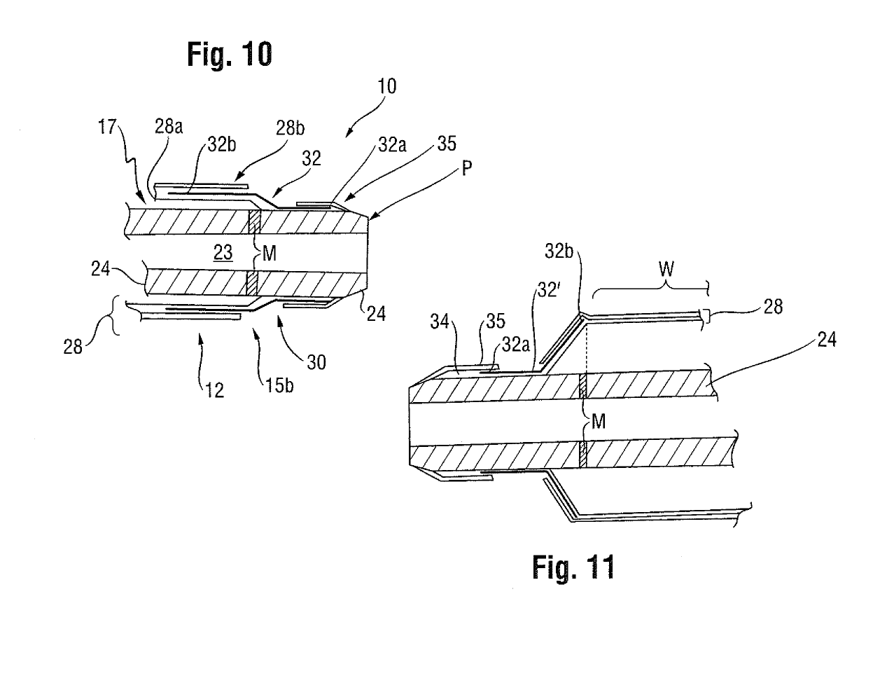

In order to provide for enhanced locatability during an interventional procedure, the catheter 10 may be adapted to ensure the alignment between the working surface W and any radiopaque identifier, such as for example the marker bands M on the portion of the shaft 24 passing within the interior of the balloon 12. In one embodiment, this is achieved using a mechanical arrangement for defining the position of one or both ends of the working surface W on inflation of the balloon. For instance, FIGS. 10 and 11 illustrate an embodiment of a catheter 10 including a balloon 12 carried on a shaft 14 in an inflated and deflated condition. An actuator 30 may be connected at one end to the balloon 12 and at another end to a separate structure, such as the tube 14 or shaft 24. For instance, the actuator 30 may be secured at the distal end 15b of the balloon wall 28, such as between an inner layer 28a and a tying layer 28b of the portion of the wall that forms the conical section 20 of the inflated balloon 12.

In one embodiment, the actuator 30 includes one or more biasing elements, such as springs. The springs may take the form of elongated, thin springs, such as for example an elongated leaf spring 32 that is bent along its longitudinal axis to “snap” between a first, home position and a second, actuated position. As a result, the actuator 30 is adapted for moving in a binary fashion between the first position corresponding to the deflated condition of the balloon 12 (FIG. 10) and the second position corresponding to the inflated condition (FIG. 11).

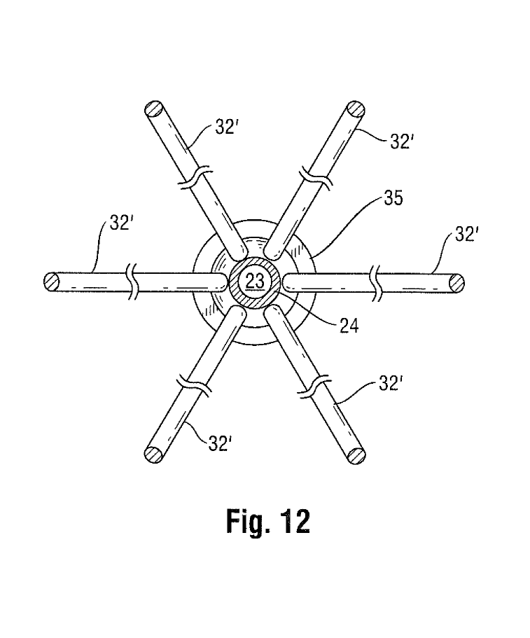

When the balloon is deflated, as shown in FIG. 10, the actuator 30 may be in a first, non-actuated condition, and lying substantially flat in order to provide the desired low profile for advancing the balloon 12 through the vessel. On inflation, and as shown in FIGS. 11 and 12, the actuator 30 moves to assume a second, pre-determined position (for spring 32, denoted as 32′), coincident with the erection of the balloon wall 28 (and, in particular, the cone section 20). The fixed second position of the actuator(s) 30 may result in the distal edge or boundary of the working surface W being precisely positioned in alignment with a radiopaque identifier, such as a marker M on the shaft 24.

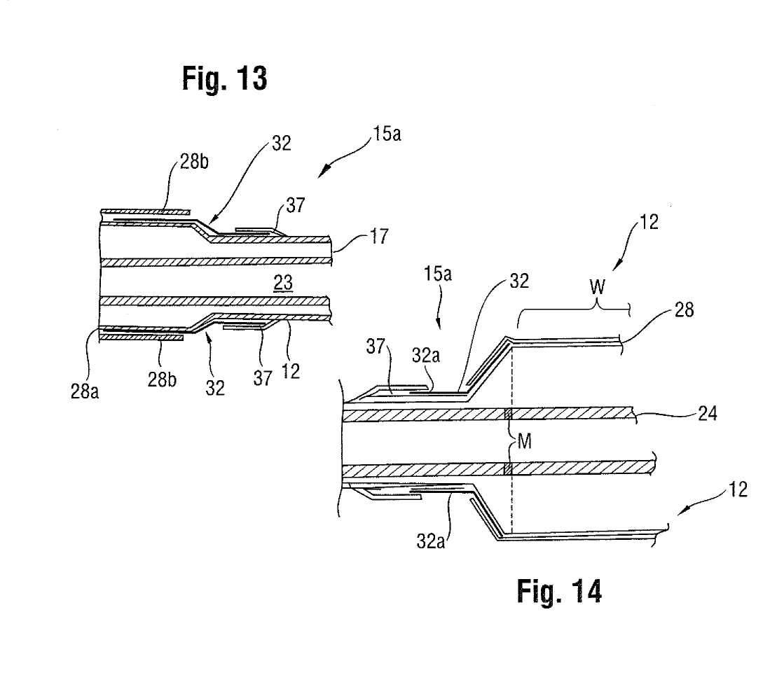

A similar arrangement may also be provided at the proximal end 15a of the balloon 12, as shown in FIGS. 13 and 14. In this way, the position of the working surface W may be accurately identified during an interventional procedure by the known alignment with the any radiopaque identifiers intended to correspond to the working surface W, such as markers M on the shaft 24, even upon inflation of the balloon 12.

Moreover, the presence of the actuator(s) 30 also helps to prevent the flattening or pancaking of the balloon 12 on deflation. This is because the folding caused by the evacuation of the contrast agent would cause the actuators 30 to move to the first position, thus helping to ensure the folding of the balloon 12 in the desired manner.

The actuator 30 may be connected in a manner that allows for movement relative to the tip P in moving between the positions. This floating arrangement in one embodiment may be achieved by providing a recess 34 in an extension 35 of the tip P at the distal end 15b of the balloon 12 for receiving and capturing the corresponding free ends 32a of the springs 32, and a corresponding recess 37 adjacent the proximal end 15a of the balloon 12. A single continuous recess may be provided, or individual recesses may be formed as pockets for receiving the individual ends 32a of the springs 32. However, it is possible to reverse the arrangement, such that the end 32a of the spring 32 at the distal end is fixed, and the end 32b corresponding to the balloon 12 is adapted for relative movement in the longitudinal direction.

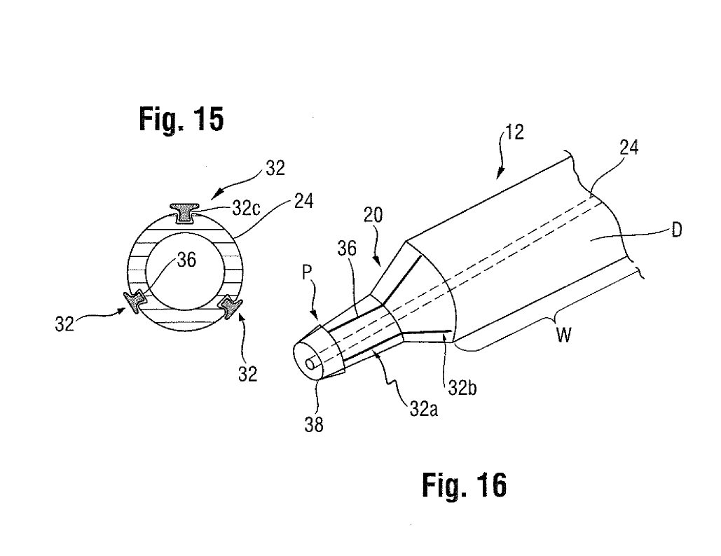

In an alternative embodiment, the actuators 30, such as springs 32, may be arranged in a similar manner, but with the permitted relative movement being provided by a mated or captured arrangement. Thus, as shown in FIGS. 15 and 16, the springs 32 may include a distal end 32a having a T-shaped cross-section, and thus having a depending portion 32c (which may form a tongue-in-groove type of arrangement, with a slight taper and a matching arrangement on the groove). This depending portion 32c is adapted for slideably engaging an elongated groove or channel 36 formed along the end of the shaft 24, distally of the cone section 20 of the balloon 12. Specifically, a wall of the shaft 24 may have the channel 36 formed along an outer surface in the longitudinal direction.

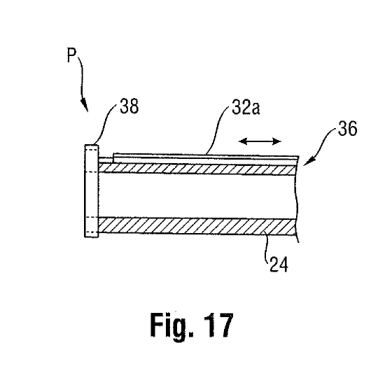

With reference to FIG. 17, it may be understood that the arrangement may create a sliding joint. The joint is arranged sufficient clearance such that the captured portion of the spring 32 may freely move within the groove or channel 36, subject to a stop, which may be provided by a cap 38 on the distal tip P of the catheter 10. The proximal end 32b of each spring 32 may be fixed to the cone section 20 of the balloon 12, as shown in FIGS. 10 and 11.

Instead of or in addition to using traditional markers in the embodiments of FIGS. 10-13, it may also be possible to instead make the actuator 30, such as spring 32, in a manner such that it is fully or partially radiopaque, so that it thus becomes the radiopaque identifier. This could be done for the embodiment where the proximal ends of the springs 32 are fixed to the balloon 12, such as in FIGS. 10 and 11. Consequently, the spring 32 on actuation is coincident with the extent of the working surface W and identifiable in view of its radiopaque quality.

Although mention is made of the use of one or more markers M on the shaft 24 to create the radiopaque identifier, it should be appreciated that the disclosure is not limited to any particular form of identifier. Indeed, the radiopaque identifier may be provided independent of the shaft 24, as disclosed in a patent application entitled, “MEDICAL BALLOON INCLUDING RADIOPAQUE INSERT FOR PRECISELY IDENTIFYING A WORKING SURFACE LOCATION,” listing as inventors Pat Byrne, Sean Wall, Angela Jensen, Andrew Schaffer, and Angela Crall, U.S. Patent App. Ser. No. 61/608,902, the disclosure of which is incorporated herein by reference.

Balloons 12 that carry one or more surface elements, such as a payload (drug, stent, or both) or a working implement (cutter, focused force wire, or the like) into the vasculature may also benefit from the foregoing description of marking techniques. For example, as shown in FIG. 16, a balloon 12 including a defined working surface W may include a portion coated with such a drug D, such as one designed for achieving a desired therapeutic effect when applied to the interior of the vessel. The drug D may be applied to the inflated balloon as part of the manufacturing process, and prior to folding for insertion in the vasculature. The clinician may thus with the benefit of a fluoroscope determine the precise positioning of the working surface W prior to inflating the balloon 12 in the vasculature to deliver the drug D to the desired location and provide the desired treatment regimen.

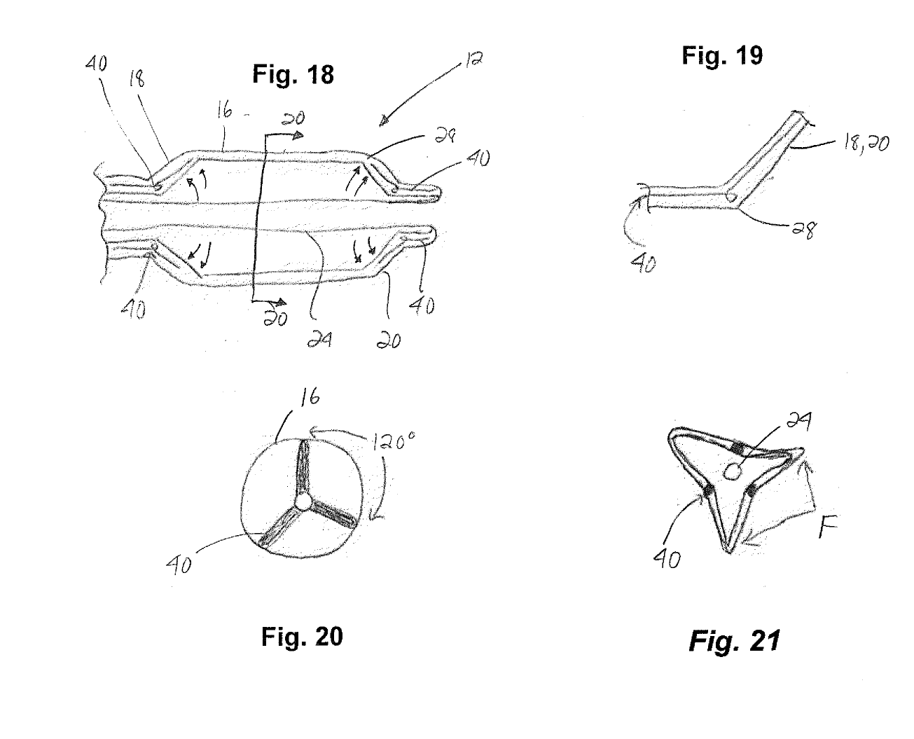

An actuator may also be used to cause the balloon 12 to collapse to a folded or partially folded state in a pre-determined or preferential manner. Thus, with reference to FIGS. 18-22, the balloon wall 28 may include one or more actuators in the form of springs. The springs may comprise torsion springs 40 secured to the inside or outside of the balloon wall 28. In one particular embodiment, these torsion springs 40 are provided in the portion of the wall 28 forming one or both of the cone sections 18, 20, and may be embedded therein. In a particular embodiment, the spring or springs 40 may be provided in only the proximal cone section 18, since this is the part that encounters the sheath upon being retracted.

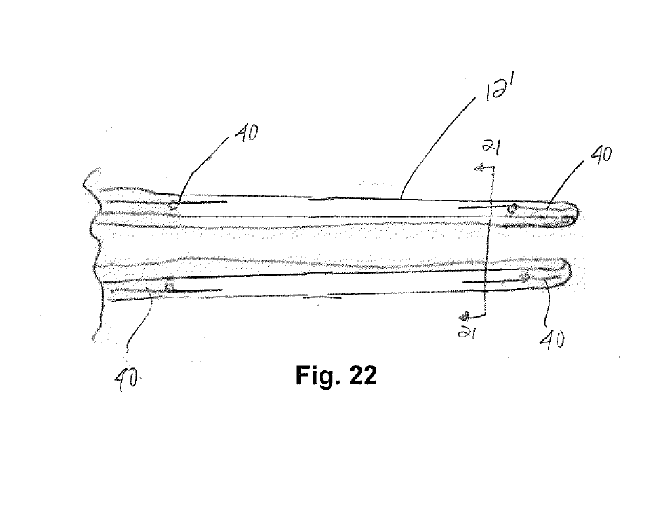

In any case, when the balloon 12 is inflated, as shown in FIG. 18, one leg of the spring 40 may extend along the portion of the wall 28 forming the conical section 18 or 20 of the balloon 12. The other leg may extend or project along an adjacent portion of the balloon 12 extending generally parallel to a longitudinal axis of the tube 24. When the balloon 12 is deflated or folded (FIGS. 21-22), the torsion springs 40 lie substantially flat, and thus do not significantly increase the profile of the balloon 12 in the folded state.

In the illustrated embodiment, as perhaps best understood with reference to FIGS. 20 and 21, three springs 40 are provided in the wall 28. The springs 40 may be spaced equidistantly apart at 120 degree intervals. However, the relative spacing and hence relative angles could be different, as could the number of springs provided, any of which variables may be adjusted depending on the particular application.

The springs 40 may be biased to provide a force in a direction toward the center of the balloon 12, such as toward tube 24, and thus may urge the balloon 12 to a folded or uninflated condition. When the balloon 12 is inflated, as shown in FIG. 18, the pressure is sufficient to overcome the spring force of the spring 40, and thus allow for the desired expansion to the unfolded condition (such as for purposes of dilatation). However, when the pressure against the wall is withdrawn, the springs 40 urge the wall 28 to collapse, returning the balloon 12 to a folded state (12′) in a particular manner (e.g., with a tendency for wings or flutes F to form (three in the embodiment with three springs) adjacent to the folds thus formed). This helps to ensure that the balloon 12 folds in a most reliable manner, and thus helps with the repositioning of the deflated balloon (including during any refolding or withdrawal step). However, since the move to the folded state is temporary, re-introducing inflation fluid and the corresponding pressure created would thus overcome the biasing force and inflate the balloon 12 again.

The torsion spring or springs 40 may be made of stainless steel or polymer materials, such as urethane. Shape memory materials that change orientation due to changes in ambient conditions may also be used to help the spring or springs 40 facilitate the folding and unfolding process. The spring or springs 40 may also be made of a bioresorbable or biodegradable material, which may be desirable when placed on an external surface of the balloon wall 28.

The following items also relate to the invention:

1. A balloon catheter, comprising:
an inflatable balloon; and
a shaft for carrying the balloon, the shaft including at least one channel formed in an outer portion of a wall of the shaft; and
an actuator having a first end connected to the balloon and a second end at least partially positioned in the channel.

2. The balloon catheter of item 1, comprising:
a shaft for carrying the balloon, the shaft including a plurality of channels formed in an outer portion of the wall of the shaft.

3. The catheter of item 2, further including an actuator having a first end connected to the balloon and a second end positioned in at least one of the channels.

4. The catheter of any of the foregoing items, comprising:
a spring connected to a wall of the balloon.

5. The catheter of item 4, wherein the spring is at least partially radiopaque.

6. The catheter of item 4 or 5, wherein the spring is connected to a conical section of the wall of the balloon.

7. The balloon catheter of any of the foregoing items, comprising:
a shaft extending in a longitudinal direction, said shaft having a proximal end and a distal end, and supporting at least one radiopaque identifier;
an inflatable balloon supported along the distal end of the shaft, the balloon when inflated including a working surface; and
an actuator for aligning at least one end of the working surface with the at least one radiopaque identifier.

8. The catheter of item 7, wherein the actuator includes a first position corresponding to a deflated state of the balloon and a second position corresponding to the inflated state of the balloon.

9. The catheter of item 7 or 8, wherein the actuator comprises a spring.

10. The catheter of any of the foregoing items 7 to 9, wherein the spring comprises a leaf spring.

11. The catheter of any of the foregoing items 7 to 10, wherein the actuator comprises a plurality of springs spaced circumferentially about the catheter.

12. The catheter of any of the foregoing items 7 to 11, wherein a first portion of the actuator is fixed to the balloon and a second portion of the actuator is adapted for movement relative to the shaft.

13. The catheter of item 12, wherein the first portion of the actuator is captured between two layers on the wall of the balloon.

14. The catheter of item 12 or 13, wherein the shaft includes a channel for at least partially receiving the second portion of the actuator.

15. The catheter of any of the foregoing items 7 to 14, further including a stop for stopping the movement of the actuator.

16. The catheter of any of the foregoing items 7 to 15, wherein the radiopaque identifier comprises a marker attached to the shaft.

17. The catheter of any of the foregoing items 7 to 16, wherein the radiopaque identifier comprises an insert positioned within the interior compartment of the balloon.

18. The catheter of any of the foregoing items 7 to 17, wherein the actuator is a first actuator for aligning a distal end of the working surface with the radiopaque identifier, and further including a second actuator for aligning a proximal end of the working surface with the radiopaque identifier.

19. The catheter of item 18, wherein each of the first and second actuators comprise a plurality of springs.

20. The catheter of any of the foregoing items 7 to 19, wherein the radiopaque identifier comprises a first marking and a second marking, and wherein the actuator is a first actuator for aligning a distal end of the working surface with the first marking, and further including a second actuator for aligning a proximal end of the working surface with the second marking.

21. The balloon catheter of any of the foregoing items, comprising:
a shaft extending in a longitudinal direction, said shaft having a proximal end and a distal end, and supporting first and second radiopaque identifiers;
a first actuator for aligning a first end of the working surface with the first radiopaque marking; and
a second actuator for aligning a second end of the working surface with the second radiopaque identifier.

22. The balloon catheter of any of the foregoing items, wherein the balloon includes a drug.

23. A method of assisting in inflating and/or deflating a balloon having a wall associated with a catheter, comprising actuating a spring connected to the balloon to assist in moving at least a portion of the balloon to an erected state and/or a deflated state.

The following items also relate to the invention:

1. A catheter, comprising:
an inflatable balloon; and
a spring connected to a wall of the balloon.

2. The catheter of item 1, wherein the spring is at least partially radiopaque.

3. The catheter of item 1 or 2, wherein the spring is connected to a conical section of the wall of the balloon.

4. The balloon catheter of any of the foregoing items, comprising:
a shaft for carrying the balloon, the shaft including at least one channel formed in an outer portion of a wall of the shaft; and
an actuator having a first end connected to the balloon and a second end at least partially positioned in the channel.

5. The balloon catheter of any of the foregoing items, comprising:
a shaft for carrying the balloon, the shaft including a plurality of channels formed in an outer portion of the wall of the shaft.

6. The catheter of item 5, further including an actuator having a first end connected to the balloon and a second end positioned in at least one of the channels.

7. The balloon catheter of any of the foregoing items, comprising:
a shaft extending in a longitudinal direction, said shaft having a proximal end and a distal end, and supporting at least one radiopaque identifier;
an inflatable balloon supported along the distal end of the shaft, the balloon when inflated including a working surface; and
an actuator for aligning at least one end of the working surface with the at least one radiopaque identifier.

8. The catheter of item 7, wherein the actuator includes a first position corresponding to a deflated state of the balloon and a second position corresponding to the inflated state of the balloon.

9. The catheter of item 7 or 8, wherein the actuator comprises a spring.

10. The catheter of any of the foregoing items 7 to 9, wherein the spring comprises a leaf spring.

11. The catheter of any of the foregoing items 7 to 10, wherein the actuator comprises a plurality of springs spaced circumferentially about the catheter.

12. The catheter of any of the foregoing items 7 to 11, wherein a first portion of the actuator is fixed to the balloon and a second portion of the actuator is adapted for movement relative to the shaft.

13. The catheter of item 12, wherein the first portion of the actuator is captured between two layers on the wall of the balloon.

14. The catheter of item 12 or 13, wherein the shaft includes a channel for at least partially receiving the second portion of the actuator.

15. The catheter of any of the foregoing items 7 to 14, further including a stop for stopping the movement of the actuator.

16. The catheter of any of the foregoing items 7 to 15, wherein the radiopaque identifier comprises a marker attached to the shaft.

17. The catheter of any of the foregoing items 7 to 16, wherein the radiopaque identifier comprises an insert positioned within the interior compartment of the balloon.

18. The catheter of any of the foregoing items 7 to 17, wherein the actuator is a first actuator for aligning a distal end of the working surface with the radiopaque identifier, and further including a second actuator for aligning a proximal end of the working surface with the radiopaque identifier.

19. The catheter of item 18, wherein each of the first and second actuators comprise a plurality of springs.

20. The catheter of any of the foregoing items 7 to 19, wherein the radiopaque identifier comprises a first marking and a second marking, and wherein the actuator is a first actuator for aligning a distal end of the working surface with the first marking, and further including a second actuator for aligning a proximal end of the working surface with the second marking.

21. The balloon catheter of any of the foregoing items, comprising:
a shaft extending in a longitudinal direction, said shaft having a proximal end and a distal end, and supporting first and second radiopaque identifiers;
a first actuator for aligning a first end of the working surface with the first radiopaque marking; and
a second actuator for aligning a second end of the working surface with the second radiopaque identifier.

22. A method of assisting in inflating and/or deflating a balloon having a wall associated with a catheter, comprising actuating a spring connected to the balloon to assist in moving at least a portion of the balloon to an erected state and/or a deflated state.

23. The balloon catheter of any of the foregoing items, wherein the balloon includes a drug.

The following items also relate to the invention:

1. A balloon catheter, comprising:
an inflatable balloon; and
a shaft for carrying the balloon, the shaft including a plurality of channels formed in an outer portion of the wall of the shaft.

2. The catheter of item 1, further including an actuator having a first end connected to the balloon and a second end positioned in at least one of the channels.

3. The balloon catheter of any of the foregoing items, comprising:
a shaft for carrying the balloon, the shaft including at least one channel formed in an outer portion of a wall of the shaft; and
an actuator having a first end connected to the balloon and a second end at least partially positioned in the channel.

4. The catheter of any of the foregoing items, comprising:
a spring connected to a wall of the balloon.

5. The catheter of item 4, wherein the spring is at least partially radiopaque.

6. The catheter of item 4 or 5, wherein the spring is connected to a conical section of the wall of the balloon.

7. The balloon catheter of any of the foregoing items, comprising:
a shaft extending in a longitudinal direction, said shaft having a proximal end and a distal end, and supporting at least one radiopaque identifier;
an inflatable balloon supported along the distal end of the shaft, the balloon when inflated including a working surface; and
an actuator for aligning at least one end of the working surface with the at least one radiopaque identifier.

8. The catheter of item 7, wherein the actuator includes a first position corresponding to a deflated state of the balloon and a second position corresponding to the inflated state of the balloon.

9. The catheter of item 7 or 8, wherein the actuator comprises a spring.

10. The catheter of any of the foregoing items 7 to 9, wherein the spring comprises a leaf spring.

11. The catheter of any of the foregoing items 7 to 10, wherein the actuator comprises a plurality of springs spaced circumferentially about the catheter.

12. The catheter of any of the foregoing items 7 to 11, wherein a first portion of the actuator is fixed to the balloon and a second portion of the actuator is adapted for movement relative to the shaft.

13. The catheter of item 12, wherein the first portion of the actuator is captured between two layers on the wall of the balloon.

14. The catheter of item 12 or 13, wherein the shaft includes a channel for at least partially receiving the second portion of the actuator.

15. The catheter of any of the foregoing items 7 to 14, further including a stop for stopping the movement of the actuator.

16. The catheter of any of the foregoing items 7 to 15, wherein the radiopaque identifier comprises a marker attached to the shaft.

17. The catheter of any of the foregoing items 7 to 16, wherein the radiopaque identifier comprises an insert positioned within the interior compartment of the balloon.

18. The catheter of any of the foregoing items 7 to 17, wherein the actuator is a first actuator for aligning a distal end of the working surface with the radiopaque identifier, and further including a second actuator for aligning a proximal end of the working surface with the radiopaque identifier.

19. The catheter of item 18, wherein each of the first and second actuators comprise a plurality of springs.

20. The catheter of any of the foregoing items 7 to 19, wherein the radiopaque identifier comprises a first marking and a second marking, and wherein the actuator is a first actuator for aligning a distal end of the working surface with the first marking, and further including a second actuator for aligning a proximal end of the working surface with the second marking

21. The balloon catheter of any of the foregoing items, comprising:
a shaft extending in a longitudinal direction, said shaft having a proximal end and a distal end, and supporting first and second radiopaque identifiers;
a first actuator for aligning a first end of the working surface with the first radiopaque marking; and
a second actuator for aligning a second end of the working surface with the second radiopaque identifier.

22. The balloon catheter of any of the foregoing items, wherein the balloon includes a drug.

23. A method of assisting in inflating and/or deflating a balloon having a wall associated with a catheter, comprising actuating a spring connected to the balloon to assist in moving at least a portion of the balloon to an erected state and/or a deflated state.

The subject matter of each of the paragraphs below citing a balloon or a catheter can be part of a balloon or a catheter respectively that is cited in any of the other paragraphs:
1.1 A balloon catheter, comprising: an elongated, tubular shaft extending in a longitudinal direction, said shaft having a proximal end and a distal end; and an inflatable balloon supported along the distal end of the shaft, the balloon when inflated including first and second spaced conical end sections and a working surface between the conical sections, the balloon further including at least one radiopaque marking identifying the transition from the conical end section to the working surface.
1.2 The catheter of paragraph 1.1, wherein the at least one radiopaque marking comprises a first radiopaque marking at a first transition between the first conical end section and the working surface, and further including a second radiopaque marking at a second transition between the second conical end section and the working surface.
1.3 The catheter of any of the foregoing paragraphs, wherein the at least one marking comprises a strip.
1.4 The catheter of any of the foregoing paragraphs, further including a plurality of radiopaque markings in the form of strips.
1.5 The catheter of paragraph 1.4, wherein the strips extend at least partially in a longitudinal direction between the first and second conical end sections.
1.6 The catheter of paragraphs 1.4 or 1.5, wherein the strips comprise annular bands.
1.7 The catheter of any of the foregoing paragraphs, wherein at least two spaced radiopaque markings are provided on each conical end section, including one adjacent a distal portion and a proximal portion of each conical end section.
1.8 The catheter of any of the foregoing paragraphs, wherein the balloon includes a barrel section between the first and second conical end sections, and further including a plurality of radiopaque markings on the barrel section.
1.9 The catheter of any of the foregoing paragraphs, wherein the marking comprises a first pattern on the conical end sections and further including a second, different pattern on the working surface.
1.10 The catheter of any of the foregoing paragraphs, wherein the at least one marking is selected from the group consisting of a pattern, a strip, a brand, a logo, a letter, a number, a word, or combinations thereof.
1.11 The catheter of any of the foregoing paragraphs, wherein the identifier comprises a scale.
1.12 The catheter of any of the foregoing paragraphs, wherein the balloon includes a drug.
1.13 The catheter of paragraph 1.12, wherein the drug corresponds to the location of the radiopaque marking.
1.14 The catheter of paragraph 1.12, wherein the drug corresponds to other than the location of the radiopaque marking.
1.15 The catheter of paragraph 1.12, wherein the radiopaque marking comprises the drug formulated to include a radiopacifier.
1.16 A balloon having a drug carried on a working surface of the balloon wall and a radiopaque identifier identifying the location of the drug on the balloon.
1.17 The balloon of paragraph 1.16, wherein the radiopaque identifier comprises a radiopaque material mixed with a formulation comprising the drug.
1.18 The balloon of paragraph 1.16, wherein the working surface is along a barrel section of the balloon, and the radiopaque identifier is on one or both cone sections of the balloon.
2.1 A balloon catheter, comprising: an elongated, tubular shaft extending in a longitudinal direction, said shaft having a proximal end and a distal end; and an inflatable balloon supported along the distal end of the shaft, the balloon when inflated including a generally cylindrical barrel section forming a working surface, and generally conical end sections that do not form a part of the working surface, the balloon further including at least one radiopaque identifier for indicating the relative position of the working surface, said identifier being provided on at least one of the conical end sections of the balloon so as to define the extent of the working surface.
2.2 The catheter of paragraph 2.1, wherein the identifier comprises a marking.
2.3 The catheter of paragraph 2.1 or 2.2, wherein a first marking is provided at a first transition between the first conical section end section and the working surface and a second marking is provided at a second transition between the second end section and the working surface.
2.4 The catheter of paragraph 2.2 or 2.3, wherein the marking comprises a strip.
2.5 The catheter of any of the foregoing paragraphs, wherein the identifier comprises a longitudinal strip extending between an end of the balloon and the barrel section.
2.6 The catheter of any of the foregoing paragraphs, further including a plurality of identifiers.
2.7 The catheter of paragraph 2.6, wherein each of the plurality of identifiers comprises a longitudinally extending strip.
2.8 The catheter of paragraph 2.6 or 2.7, wherein the identifiers comprise annular bands.
2.9 The catheter of paragraph 2.6 or paragraph 2.8 as dependent on paragraph 2.6, wherein the identifiers comprise longitudinally extending strips.
2.10 The catheter of any of the foregoing paragraphs 2.1 to 2.9, wherein at least two spaced radiopaque identifiers are provided on each end section.
2.11 The catheter of any of the foregoing paragraphs 2.1 to 2.10, further including at least one radiopaque identifier on the barrel section.
2.12 The catheter of any of the foregoing paragraphs 2.1 to 2.11, wherein the identifier is a first identifier comprising a first pattern, and further including a second identifier comprising a second, different pattern.
2.13 The catheter of any of the foregoing paragraphs 2.1 to 2.12, wherein the identifier includes at least one letter or number.
2.14 The catheter of any of the foregoing paragraphs 2.1 to 2.13, wherein the identifier comprises a logo.
2.15 The catheter of any of the foregoing paragraphs 2.1 to 2.14, wherein the identifier comprises a scale.
2.16 The catheter of any of the foregoing paragraphs 2.1 to 2.15, further including a drug on the balloon.
3.1 An inflatable balloon for use in connection with a catheter, comprising: an inflatable body including a working surface extending in a longitudinal direction between a first end and a second end, the body having at least one radiopaque identifier provided along the body for identifying at least a first end of the working surface, the radiopaque identifier having a first radiographic quality for identifying the location of the first end of the working surface and a second radiographic quality at a location other than at the first end of the working surface.
3.2 The balloon of paragraph 3.1, wherein the second radiographic quality is provided for identifying the second end of the working surface.
3.3 The catheter of paragraph 3.2, wherein the first radiographic quality and the second radiographic quality are substantially the same.
3.4 The balloon of paragraph 3.1, wherein the radiopaque identifier comprises a marking.
3.5 The balloon of paragraph 3.1, wherein the radiopaque identifier follows a generally helical path from the first end to the second end of the working surface.
3.6 The balloon of paragraph 3.1, wherein the identifier comprises a plurality of helical identifiers extending along the working surface.
3.7 The balloon of paragraph 3.1, wherein the identifier comprises a radiopaque filament.
3.8 The balloon of paragraph 3.7, wherein the filament is wound helically along at least a portion of the working surface of the balloon.
3.9 The balloon of any of the foregoing paragraphs 3.1 to 3.8, further including a drug on the balloon.
3.10 A balloon for use in connection with a catheter, comprising: a body having an outer surface and at least one winding extending along the outer surface of the balloon, said balloon having a radiopaque quality.
3.11 The balloon of paragraph 3.10, wherein the winding comprises a radiopaque filament.
3.12 The balloon of any of the foregoing paragraphs, wherein the radiopaque identifier comprises a helical pattern or a diamond pattern.
3.13 A catheter including the balloon of any of the foregoing paragraphs.
3.14 An inflatable balloon for use in connection with a catheter comprising a radiopaque identifier comprising a helical pattern or a diamond pattern.
4.1 A balloon catheter for use in connection with a guidewire, comprising: an elongated, tubular shaft extending in a longitudinal direction, said shaft having a proximal end and a distal end; an inflatable balloon supported along the distal end of the shaft, the balloon when inflated including first and second spaced ends and a working surface between the ends; and at least one wire including at least a radiopaque portion for identifying the location of working surface of the balloon.
4.2 The catheter of paragraph 4.1, wherein said wire comprises a material having a shape memory for adjusting between a first state and a second state.
4.3 The catheter of paragraph 4.1 or 4.2, wherein the at least one wire extends generally in the longitudinal direction.
4.4 The catheter of any of the foregoing paragraphs 4.1 to 4.3, wherein the radiopaque portion is elongated.
4.5 The catheter of any of the foregoing paragraphs 4.1 to 4.4, wherein the wire at least partially comprises a polymer.
4.6 The catheter of any of the foregoing paragraphs 4.1 to 4.5, wherein the at least one wire is at least partially elastic.
4.7 The catheter of any of the foregoing paragraphs 4.1 to 4.6, comprising: a plurality of wires extending generally in the longitudinal direction, at least one of the wires including at least a radiopaque portion for identifying the location of working surface of the balloon.
4.8 The catheter of any of the foregoing paragraphs 4.1 to 4.7, wherein at least one wire extends along an outer surface of the balloon.
4.9 The catheter of any of the foregoing paragraphs 4.1 to 4.8, wherein at least one wire extends along an inner surface of the balloon.
4.10 The catheter of any of the foregoing paragraphs 4.1 to 4.9, wherein at least one wire extends from the first end to the second end of the balloon.
4.11 The catheter of any of the foregoing paragraphs 4.1 to 4.10, wherein the radiopaque portion of at least one wire extends along a portion of the balloon corresponding to the working surface.
4.12 The catheter of any of the foregoing paragraphs 4.1 to 4.11, wherein the radiopaque portion of at least one wire extends along other than along the portion of the balloon corresponding to the working surface.
4.13 The catheter of paragraph 4.7 or any of paragraphs 4.8 to 4.12 as dependent on paragraph 4.7, wherein the wires are spaced substantially equidistantly around a circumference of the balloon.
4.14 The catheter of any of the foregoing paragraphs 4.1 to 4.13, wherein the wire includes a compliant or semi-compliant portion.
4.15 The catheter of any of the foregoing paragraphs 4.1 to 4.14, wherein at least one end of the at least partially radiopaque wire is attached to a bond connecting the balloon to the shaft.
4.16 The catheter of any of the foregoing paragraphs 4.1 to 4.15, further including a drug provided on the balloon.
4.17 The catheter of any of the foregoing paragraphs 4.1 to 4.16, wherein at least one wire at least partially comprises a material having a shape memory for adjusting between a first state and a second state.
4.18 The catheter of paragraph 4.2 or 4.17, wherein the shape memory material comprises NITINOL.
5.1 A balloon catheter adapted for use with a guidewire, comprising: an elongated, tubular shaft extending in a longitudinal direction, said shaft having a proximal end and a distal end; an inflatable balloon supported along the distal end of the shaft, the balloon when inflated including first and second spaced ends and a working surface between the ends; and an insert located within the interior compartment of the balloon, the insert including at least a radiopaque portion separate from the shaft.
5.2 The catheter of paragraph 5.1, wherein the insert is adapted for moving relative to the shaft.
5.3 The catheter of paragraph 5.1 or 5.2, wherein the insert extends from a first end of the balloon to one end of the working surface.
5.4 The catheter of any of the foregoing paragraphs 5.1 to 5.3, wherein the insert comprises a tube made at least partially of a radiopaque material.
5.5 The catheter of any of the foregoing paragraphs 5.1 to 5.4, wherein the insert comprises at least one finger.
5.6 The catheter of paragraph 5.5, wherein the finger includes a radiopaque end portion.
5.7 The catheter of any of the foregoing paragraphs 5.1 to 5.6, wherein the insert comprises a plurality of fingers adapted for moving from a retracted condition to an expanded condition when the balloon is inflated.
5.8 The catheter of any of the foregoing paragraphs 5.1 to 5.7, further including a retractable sheath at least partially covering the insert.
5.9 The catheter of any of the foregoing paragraphs 5.1 to 5.8, wherein the insert comprises a wire.
5.10 The catheter of paragraph 5.9, wherein the wire includes a radiopaque portion corresponding to the working surface.
5.11 The catheter of paragraph 5.10, wherein the wire extends from the first end to the second end of the balloon, and the radiopaque portion comprises an intermediate portion of the wire.
5.12 The catheter of paragraph 5.10 or 5.11, wherein the wire extends from the first end to the second end of the balloon, and the radiopaque portion comprises an end portion of the wire.
5.13 The catheter of any of the foregoing paragraphs 5.1 to 5.12, wherein at least one end of the insert is connected at a location where the balloon connects to the tubular shaft.
5.14 The catheter of any of the foregoing paragraphs 5.1 to 5.13, wherein the insert comprises an annular band.
5.15 The catheter of any of the foregoing paragraphs 5.1 to 5.14, wherein the insert includes perforations.
5.16 The catheter of any of the foregoing paragraphs 5.1 to 5.15, wherein the insert comprises a material having a shape memory.
5.17 The catheter of any of the foregoing paragraphs 5.1 to 5.16, further including a drug on the balloon.
6.1 A parison for being blow molded into a medical balloon for a catheter, comprising: a first tubular layer having a functional modification; and a second tubular layer adapted for bonding with the first tubular layer to form the blow molded balloon.
6.2 The parison of paragraph 6.1, wherein the first layer is external to the second layer.
6.3 The parison of paragraph 6.1, wherein the first layer is internal to the second layer.
6.4 The parison of any of the foregoing paragraphs, wherein the functional modification comprises a radiopaque strip.
6.5 The parison of paragraph 6.4, wherein the strip comprises a circumferential band.
6.6 The parison of paragraph 6.4 or 6.5, wherein the strip extends between a first end and a second end of the first layer.
6.7 The parison of any of the foregoing paragraphs, wherein the first tubular layer is spaced from the second tubular layer.
6.8 The parison of any of the foregoing paragraphs, wherein the functional modification is selected from the group consisting of an added radiopacifier, a surface pattern, an etching, one or more perforations, and combinations of the foregoing.
6.9 A medical balloon formed by the parison of any of the foregoing paragraphs, comprising: a tubular, inflatable body comprising a wall, the body including first and second generally conical ends and a generally cylindrical barrel section between the generally conical ends and providing a working surface.
6.10 The balloon of paragraph 6.9, wherein the first layer extends from the first end to the second end of the balloon.
6.11 The balloon of paragraph 6.9, wherein the first layer extends along only the working surface.
6.12 The balloon of any of paragraphs 6.9 to 6.11, wherein the first layer extends along an entire circumference of a portion of the wall.
6.13 The balloon of any of paragraphs 6.9 to 6.12, wherein the first layer extends along the full circumference of the wall.
6.14 The balloon of any of paragraphs 6.9 to 6.13, wherein the wall includes first and second spaced shoulders, and wherein the first layer is positioned between the shoulders.
6.15 The balloon of any of paragraphs 6.9 to 6.14, wherein the first and second layers both extend from a first end to a second end of the balloon.
6.16 The balloon of any of paragraphs 6.9 to 6.15, further comprising an at least partially radiopaque tube positioned over the barrel section and extending substantially along the working surface.
6.17 The balloon of paragraph 6.16, further including first and second shoulders adjacent the proximal and distal ends of the radiopaque tube.
6.18 The balloon of paragraph 6.16 or 6.17, wherein the entire tube is radiopaque.
7.1 A balloon catheter, comprising: an elongated, tubular shaft having a proximal end and a distal end; and a balloon positioned along the distal end of the shaft, a portion of a wall of the balloon partially comprising a coextruded radiopaque material.
7.2 The catheter of paragraph 7.1, wherein the radiopaque portion comprises at least one strip extending along a working surface of the balloon.
7.3 The catheter of paragraph 7.1 or 7.2, wherein the radiopaque portion comprises at least one strip extending along a full length surface of the balloon.
7.4 The catheter of any of paragraphs 7.1 to 7.3, wherein the radiopaque portion comprises at least one strip extending along a first cone section of the balloon.
7.5 The catheter of paragraph 7.4, wherein the radiopaque portion comprises at least one strip extending along a second cone section of the balloon.
7.6 The catheter of any of paragraphs 7.1 to 7.5, wherein the balloon includes a plurality of radiopaque portions.
7.7 The catheter of paragraph 7.6, wherein each of the plurality of radiopaque portions comprises a longitudinal strip.
7.8 The catheter of paragraph 7.7, wherein the strips extend at least along a working surface of the balloon.
7.9 The catheter of any of paragraphs 7.6 to 7.8, wherein the plurality of radiopaque portions are spaced apart in a circumferential direction.
7.10 The catheter of any of the foregoing paragraphs 7.1 to 7.9, wherein the balloon includes a barrel section and conical sections at each end of the barrel section, and wherein the radiopaque portion is provided on the barrel section.
7.11 The catheter of any of the foregoing paragraphs 7.1 to 7.10, wherein the balloon includes a barrel section and conical sections at each end of the barrel section, and wherein the radiopaque portion is provided on one or both of the cone sections.
7.12 The catheter of any of the foregoing paragraphs 7.1 to 7.11, wherein the radiopaque portion comprises a layer of the balloon wall.
7.13 The catheter of paragraph 7.12, wherein the layer comprises an inner layer.
7.14 The catheter of paragraph 7.12 or 7.13, wherein the layer comprises an outer layer.
7.15 The catheter of paragraph 7.14, wherein the outer layer is etched.
7.16 The catheter of any of paragraphs 7.12 to 7.15, wherein the balloon includes a barrel section and conical sections at each end of the barrel section, and the layer extends along the entire barrel section.
7.17 The catheter of any of paragraphs 7.12 to 7.16, wherein the balloon includes a barrel section and conical sections at each end of the barrel section, and the layer extends along the entirety of one or both of the conical sections.
7.18 The catheter of any of the foregoing paragraphs 7.1 to 7.17, wherein all portions of the wall comprise coextruded radiopaque material.
7.19 The catheter of any of the foregoing paragraphs 7.1 to 7.18, further including a drug on the balloon.
7.20 The catheter of any of the foregoing paragraphs 7.1 to 7.19, wherein the radiopaque material comprises ePTFE.
8.1 A balloon catheter, comprising: a shaft extending in a longitudinal direction, said shaft having a proximal end and a distal end, and supporting at least one radiopaque identifier; an inflatable balloon supported along the distal end of the shaft, the balloon when inflated including a working surface; and an actuator for aligning at least one end of the working surface with the at least one radiopaque identifier.
8.2 The catheter of paragraph 8.1, wherein the actuator includes a first position corresponding to a deflated state of the balloon and a second position corresponding to the inflated state of the balloon.
8.3 The catheter of paragraph 8.1 or 8.2, wherein the actuator comprises a spring.
8.4 The catheter of any of the foregoing paragraphs 8.1 to 8.3, wherein the spring comprises a leaf spring.
8.5 The catheter of any of the foregoing paragraphs 8.1 to 8.4, wherein the actuator comprises a plurality of springs spaced circumferentially about the catheter.
8.6 The catheter of any of the foregoing paragraphs 8.1 to 8.5, wherein a first portion of the actuator is fixed to the balloon and a second portion of the actuator is adapted for movement relative to the shaft.
8.7 The catheter of paragraph 8.6, wherein the first portion of the actuator is captured between two layers on the wall of the balloon.
8.8 The catheter of paragraph 8.6 or 8.7, wherein the shaft includes a channel for at least partially receiving the second portion of the actuator.
8.9 The catheter of any of the foregoing paragraphs 8.1 to 8.8, further including a stop for stopping the movement of the actuator.
8.10 The catheter of any of the foregoing paragraphs 8.1 to 8.9, wherein the radiopaque identifier comprises a marker attached to the shaft.
8.11 The catheter of any of the foregoing paragraphs 8.1 to 8.10, wherein the radiopaque identifier comprises an insert positioned within the interior compartment of the balloon.
8.12 The catheter of any of the foregoing paragraphs 8.1 to 8.11, wherein the actuator is a first actuator for aligning a distal end of the working surface with the radiopaque identifier, and further including a second actuator for aligning a proximal end of the working surface with the radiopaque identifier.
8.13 The catheter of paragraph 8.12, wherein each of the first and second actuators comprise a plurality of springs.
8.14 The catheter of any of the foregoing paragraphs, wherein the radiopaque identifier comprises a first marking and a second marking, and wherein the actuator is a first actuator for aligning a distal end of the working surface with the first marking, and further including a second actuator for aligning a proximal end of the working surface with the second marking.
8.15 The balloon catheter of any of the foregoing paragraphs 8.1 to 8.14, comprising: a shaft extending in a longitudinal direction, said shaft having a proximal end and a distal end, and supporting first and second radiopaque identifiers; a first actuator for aligning a first end of the working surface with the first radiopaque marking; and a second actuator for aligning a second end of the working surface with the second radiopaque identifier.
8.16 The balloon catheter of any of the foregoing paragraphs 8.1 to 8.15, comprising: a shaft for carrying the balloon, the shaft including at least one channel formed in an outer portion of a wall of the shaft; and an actuator having a first end connected to the balloon and a second end at least partially positioned in the channel.
8.17 The balloon catheter of any of the foregoing paragraphs 8.1 to 8.16, comprising: a shaft for carrying the balloon, the shaft including a plurality of channels formed in an outer portion of the wall of the shaft.
8.18 The catheter of paragraph 8.17, further including an actuator having a first end connected to the balloon and a second end positioned in at least one of the channels.
8.19 The catheter of any of the foregoing paragraphs 8.1 to 8.8, comprising: a spring connected to a wall of the balloon.
8.20 The catheter of paragraph 8.19, wherein the spring is at least partially radiopaque.
8.21 The catheter of paragraph 8.19 or 8.20, wherein the spring is connected to a conical section of the wall of the balloon.
8.22 The balloon catheter of any of the foregoing paragraphs 8.1 to 8.21, wherein the balloon includes a drug.
9.1 A balloon catheter for use with a guidewire, comprising: an elongated, tubular shaft extending in a longitudinal direction, said shaft having a proximal end and a distal end; an inflatable balloon connected to the distal end of the shaft, the balloon including a working surface; a radiopaque identifier for identifying the working surface; and a receiver adjacent the proximal end of the shaft and adapted for allowing the shaft to move relative to the receiver in at least the longitudinal direction.
9.2 The catheter of paragraph 9.1, wherein the shaft carries a stop, and the receiver further includes a recess for receiving the stop, said recess having a dimension in the longitudinal direction that is greater than a corresponding dimension of the stop.
9.3 The catheter of paragraph 9.2, further including a tube for supplying an inflation fluid to inflate the balloon, said tube being connected to the receiver and generally coaxial with the shaft, and wherein the stop forms a seal with the recess to prevent the inflation fluid from passing around the shaft.
9.4 The catheter of paragraph 9.3, wherein the seal comprises an O-ring arranged coaxially with the shaft.
9.5 The catheter of paragraph 9.1, wherein the radiopaque identifier is separate from the shaft.
9.6 The catheter of paragraph 9.5, wherein the radiopaque identifier comprises an insert positioned within the interior compartment of the balloon.
9.7 The catheter of paragraph 9.6, wherein the insert comprises a tubular sleeve arranged coaxially with the shaft.
9.8 The catheter of paragraph 9.6, wherein the insert comprises a first insert at a proximal end of the balloon and a second insert at a distal end of the balloon.
9.9 The catheter of paragraph 9.1, further including a guidewire for positioning in the shaft.
9.10 A hub for a balloon catheter having an elongated, tubular shaft extending in a longitudinal direction, said shaft having a proximal end and a distal end, and an inflatable balloon connected to the distal end of the shaft for being inflated by an inflation fluid, comprising: a body including a receiver for receiving a proximal portion of the shaft and adapted for allowing the shaft to move relative to the receiver in at least the longitudinal direction; and a stop for restraining the movement of the shaft relative to the body in the longitudinal direction.
9.11 The hub of paragraph 9.10, wherein the body includes a guidewire port arranged in communication with the receiver, and further including an inflation port for introducing the inflation fluid for inflating the balloon.
9.12 The hub of paragraph 9.10, wherein the receiver further includes a recess for receiving the stop, said recess having a dimension in the longitudinal direction that is greater than a corresponding dimension of the stop.
9.13 The hub of paragraph 9.12, wherein the stop forms a seal with the recess to prevent the inflation fluid from passing.
9.14 The hub of paragraph 9.10, wherein the stop comprises an O-ring.
9.15 A catheter including a guidewire shaft having a distal end connected to a balloon and at a proximal end mounted for sliding movement.
9.16 The catheter of any of the foregoing paragraphs, further including a drug on the balloon.
9.17 A catheter comprising a hub for receiving a proximal end of a guidewire shaft, the shaft being adapted to slideably move in a restrained manner relative to the hub.
10.1 A balloon catheter, comprising: an elongated tubular shaft having a proximal end and a distal end spaced apart in a longitudinal direction, the shaft along a distal portion including at least one radiopaque identifier, said distal portion being formed of a material resistant to elongation in the longitudinal direction; and an inflatable, non-compliant balloon extending over the distal portion of the shaft.
10.2 The catheter according to paragraph 10.1, wherein the balloon includes a generally cylindrical barrel section positioned between generally conical sections, said barrel section including a working surface having at least one edge aligned with the radiopaque identifier.
10.3 The catheter according to paragraph 10.2, wherein the radiopaque identifier comprises a first marker positioned at the at least one edge of the working surface, and further including a second marker positioned at the opposite edge of the working surface in the longitudinal direction.
10.4 The catheter according to paragraph 10.2, wherein each marker comprises a radiopaque band swaged to the distal portion of the shaft.
10.5 The catheter according to paragraph 10.1, wherein the distal portion of the shaft comprises a tube adapted for guiding a guidewire from a proximal end of the balloon to a distal end of the balloon.
10.6 The catheter according to paragraph 10.1, wherein at least the distal portion of the shaft comprises steel.
10.7 The catheter according to paragraph 10.1, wherein the shaft comprises steel.
10.8 The catheter according to paragraphs 10.6 or 10.7, wherein the steel shaft comprises a stainless steel.
10.9 The catheter according to paragraphs 10.7 or 10.8, wherein the steel shaft includes a spiral cut along a portion other than the distal portion covered by the balloon.
10.10 The catheter according to paragraphs 10.7 or 10.8, wherein the steel shaft comprises a polymer layer.
10.11 The catheter according to paragraph 10.10, wherein the polymer layer comprises an outer layer of the shaft.
10.12 The catheter according to paragraph 10.1, wherein the distal portion of the shaft comprises a polymer shaft including a braid or mesh.
10.13 The catheter according to paragraph 10.1, wherein the balloon includes a generally cylindrical barrel section positioned between generally conical sections, the distal portion of the shaft extending from a first end of a first conical section to a second end of a second conical section.
10.14 The catheter according to paragraph 10.1, wherein the non-compliant balloon comprises one or more inelastic fibers.
10.15 The catheter according to paragraph 10.1, wherein the non-compliant balloon comprises polyethylene terephthalate.
10.16 The catheter of any of the foregoing paragraphs 10.1 to 10.15, further including a drug on the balloon.
11.1 A balloon catheter, comprising: a shaft extending in a longitudinal direction and adapted for expanding from a compressed condition to an expanded condition in the longitudinal direction, the shaft supporting at least one radiopaque identifier; and an inflatable balloon positioned along the shaft, the balloon when inflated including a working surface for aligning with the radiopaque identifier in at least the expanded condition of the shaft.
11.2 The catheter of paragraph 11.1, wherein the expandable shaft comprises a first portion connected in tandem to an expandable element.
11.3 The catheter of paragraphs 11.1 or 11.2, wherein the expandable element comprises a spring.
11.4 The catheter of paragraph 11.3, wherein the spring comprises a coil spring.
11.5 The catheter of paragraphs 11.3 or 11.4, wherein the spring comprises a tension coil spring.
11.6 The catheter of paragraph 11.2, wherein the expandable element comprises a bellows.
11.7 The catheter of paragraph 11.2, wherein the expandable element comprises a fiber matrix.
11.8 The catheter of paragraph 11.7, further including a spring associated with the fiber matrix.
11.9 The catheter of any of paragraphs 11.2-11.8, wherein the expandable element is inside an interior compartment of the balloon.
11.10 The catheter of any of paragraphs 11.2-11.8, wherein the expandable element is outside an interior compartment of the balloon.
11.11 The catheter of any of paragraphs 11.2-11.10, wherein the expandable element connects to one end of the balloon.
11.12 The catheter of any of paragraphs 11.2-11.10, wherein the expandable element connects the first portion of the shaft to a second portion of the shaft.
11.13 The catheter of any of the foregoing paragraphs 11.1 to 11.12, wherein the shaft comprises an inflation lumen for delivering an inflation fluid to the balloon.
11.14 The catheter of any of the foregoing paragraphs 11.1 to 11.13, wherein the expandable shaft in at least a partially expanded condition a port for delivering the inflation fluid to the balloon, said port being closed when the shaft is in a non-expanded condition.
11.15 The catheter of any of the foregoing paragraphs 11.1 to 11.14, wherein the expandable shaft comprises a first expandable element connecting a first portion of the shaft to a second portion of the shaft, and further including a second expandable element connecting the second portion of the shaft to a third portion of the shaft.
11.16 The catheter of paragraph 11.15, wherein the first and second expandable elements comprise first and second coil springs.
11.17 The catheter of paragraph 11.16, wherein the first and second coil springs have different spring constants.
11.18 The catheter of any of the foregoing paragraphs 11.1 to 11.17, wherein the radiopaque identifier comprises a pair of spaced radiopaque markers, one positioned in alignment with a first end of the working surface and another positioned at a second end of the working surface.
11.19 The catheter of any of paragraphs 11.15-11.18, wherein the first and second expandable elements comprise a radiopaque material.
11.20 The catheter of any of the foregoing paragraphs 11.1 to 11.19, wherein the radiopaque identifier comprises a spring.
11.21 The catheter of paragraph 11.2, wherein the expandable element comprises a spring having a variable spring constant.
11.22 The catheter of any of the foregoing paragraphs 11.1 to 11.21, wherein the shaft comprises a guidewire lumen.
11.23 The catheter of any of the foregoing paragraphs 11.1 to 11.22, further including a passage adjacent the tip for receiving a guidewire external to the balloon.
11.24 The catheter of paragraph 11.2, wherein the first portion is adjacent a distal end of the shaft.
11.25 A balloon catheter, comprising: a shaft; a balloon; and an expandable element adapted for expanding in the longitudinal direction connecting the shaft to the balloon.
11.26 The catheter of paragraph 11.25, wherein the expandable element is selected from the group consisting of a spring, a bellows, a fiber matrix, or combinations of the foregoing.
11.27 The catheter of paragraph 11.25 or 26, wherein the expandable element comprises an encapsulated spring.
11.28 A balloon catheter comprising a balloon and an inflation lumen including an expandable element adapted for expanding in the longitudinal direction for providing a fluid to the balloon.
11.29 The catheter of any of paragraphs 11.25-11.28, wherein the expandable element comprises a radiopaque material.
11.30 The catheter of any of the foregoing paragraphs 111 to 11.29, further including a drug on the balloon.
12.1 A balloon catheter, comprising: an elongated, tubular shaft extending in a longitudinal direction, said shaft having a proximal end and a distal end; and a balloon having an inflation compartment formed a balloon wall including a working surface, and further including at least one chamber adjacent to the working surface adapted for receiving an identifier for identifying the location of the working surface.
12.2 The balloon catheter of paragraph 12.1, wherein the shaft includes a first lumen for supplying a fluid to the chamber.
12.3 The balloon catheter of paragraph 12.2, wherein the shaft includes a port between the first lumen and the chamber.
12.4 The balloon catheter of paragraph 12.2, wherein the shaft includes a second lumen for supplying a fluid to an interior compartment of the balloon.
12.5 The balloon catheter of paragraph 12.4, wherein the shaft includes a port between the second lumen and the interior compartment.
12.6 The balloon catheter of any of the foregoing paragraphs 12.1 to 12.5, wherein the identifier comprises a contrast agent.
12.7 The balloon catheter of any of the foregoing paragraphs 12.1 to 12.6, wherein the contrast agent comprises a material selected from the group consisting of a radiopacifier, polyvinyl acetate, cellulose, a fluid, a liquid, a solid, a powder, or combinations of the foregoing.
12.8 The balloon catheter of any of the foregoing paragraphs 12.1 to 12.7, wherein the chamber comprises a first chamber at a proximal end of the balloon, and further including a second chamber at a distal end of the balloon.
12.9 The balloon catheter of paragraph 12.8, wherein the second chamber is adapted for receiving the identifier from a lumen in the shaft in fluid communication with the first chamber via a port.
12.10 The balloon catheter of any of the foregoing paragraphs 12.1 to 12.9, wherein the chamber is generally annular.
12.11 The balloon catheter of any of the foregoing paragraphs 12.1 to 12.10, wherein the chamber is positioned between a transition from a barrel section to a conical section of the balloon and an end of the balloon.
12.12 The balloon catheter of any of the foregoing paragraphs 12.1 to 12.11, wherein the chamber is provided by a film attached to the balloon wall.
12.13 The balloon catheter of any of the foregoing paragraphs 12.1 to 12.12, wherein the chamber is embedded in the balloon wall.
12.14 The balloon catheter of any of the foregoing paragraphs 12.1 to 12.13, wherein the chamber is provided by a film extending between the balloon wall and an outer surface of the shaft.

While the disclosure presents certain embodiments to illustrate the inventive concepts, numerous modifications, alterations, and changes to the described embodiments are possible without departing from the sphere and scope of the present invention, as defined in the appended claims. For example, the ranges and numerical values provided in the various embodiments are subject to variation due to tolerances, due to variations in environmental factors and material quality, and due to modifications of the structure and shape of the balloon, and thus can be considered to be approximate and the term “approximately” means that the relevant value can, at minimum, vary because of such factors. Accordingly, it is intended that the present disclosure not be limited to the described embodiments, but that it has the full scope defined by the language of the following claims, and equivalents thereof.

1. A balloon catheter, comprising:
a shaft extending in a longitudinal direction, said shaft having a proximal end and a distal end, and supporting at least one radiopaque identifier,
an inflatable balloon supported along the distal end of the shaft, the balloon when inflated including a working surface; and
an actuator for aligning at least one end of the working surface with the at least one radiopaque identifier.

2. The catheter of claim 1, wherein the actuator includes a first position corresponding to a deflated state of the balloon and a second position corresponding to the inflated state of the balloon.

3. The catheter of claim 1, wherein the actuator comprises a spring.

4. The catheter of claim 1, wherein the spring comprises a leaf spring.

5. The catheter of claim 1, wherein the actuator comprises a plurality of springs spaced circumferentially about the catheter.

6. The catheter of claim 1, wherein a first portion of the actuator is fixed to the balloon and a second portion of the actuator is adapted for movement relative to the shaft.

7. The catheter of claim 6, wherein the first portion of the actuator is captured between two layers on the wall of the balloon.

8. The catheter of claim 6, wherein the shaft includes a channel for at least partially receiving the second portion of the actuator.

9. The catheter of claim 1, further including a stop for stopping the movement of the actuator.

10. The catheter of claim 1, wherein the radiopaque identifier comprises a marker attached to the shaft.

11. The catheter of claim 1, wherein the radiopaque identifier comprises an insert positioned within the interior compartment of the balloon.

12. The catheter of claim 1, wherein the actuator is a first actuator for aligning a distal end of the working surface with the radiopaque identifier, and further including a second actuator for aligning a proximal end of the working surface with the radiopaque identifier.

13. The catheter of claim 12, wherein each of the first and second actuators comprise a plurality of springs.

14. The catheter of claim 1, wherein the radiopaque identifier comprises a first marking and a second marking, and wherein the actuator is a first actuator for aligning a distal end of the working surface with the first marking, and further including a second actuator for aligning a proximal end of the working surface with the second marking.

15.-17. (canceled)

18. The catheter of claim 17, further including an actuator having a first end connected to the balloon and a second end positioned in at least one of the channels.

19. The catheter of claim 1, comprising:
a spring connected to a wall of the balloon.

20. The catheter of claim 19, wherein the spring is at least partially radiopaque.

21. The catheter of claim 19, wherein the spring is connected to a conical section of the wall of the balloon.

22.-31. (canceled)

32. A medical balloon comprising a wall with one or more springs embedded therein.

33.-34. (canceled)

35. A catheter, comprising:
an inflatable balloon; and
an actuator comprising a torsion spring connected to a wall of the balloon to assist in moving at least a portion of the balloon to an erected state and or a folded state.

36.-43. (canceled)
